(19) United States
(12) Patent Application Publication
(10) Pub. No.: US 2015/0101074 A1
(43) Pub. Date: Apr. 9, 2015

(54) MALE STERILE GARLIC PLANTS, HYBRID OFFSPRING OF SAME AND METHODS OF GENERATING AND USING SAME

(71) Applicants: The State of Israel, Ministry of Agriculture & Rural Development, Agriculture Research, Beit-Dagan (IL); Yissum Research Development Company of the Hebrew University of Jerusalem Ltd., Jerusalem (IL)

(72) Inventors: Rina Kamenetsky Goldstein, Rehovot (IL); Haim David Rabinowitch, Kiryat-Ono (IL); Einat Shemesh Mayer, Kiryat-Ono (IL)

(21) Appl. No.: 14/382,323

(22) PCT Filed: Feb. 28, 2013
(86) PCT No.: PCT/IL2013/050178

§ 371 Date: Sep. 1, 2014

Related U.S. Application Data
(60) Provisional application No. 61/605,228, filed on Mar. 1, 2012.

(51) Int. Cl. A01H 5/06; A01G 1/00; A01H 1/06; C12N 5/04; A01H 1/02; A01H 4/00

(52) U.S. Cl. CPC A01H 5/06; A01H 1/02; A01H 4/008; A01H 1/06; C12N 5/04; A01G 1/001
USPC 800/274; 800/303; 800/277; 800/276; 435/410; 47/58.1 FV

(57) ABSTRACT

Garlic plants and parts thereof are provided also provided are methods of generating and using same. Also provided are processed products generated from the garlic plants of parts thereof.

### FIELD AND BACKGROUND OF THE INVENTION

The present invention, in some embodiments thereof, relates to male sterile garlic plants, hybrid offspring of same and methods of generating and using same.

Pollination and fertilization are required for seed production. Cross-pollination is a major means of gene flow between populations within and between species, while in many crops, self-pollination leads to inbreeding depression with the consequent reduction in quality and yields (Jones and Davis 1944). Male sterility is a valuable resource for hybrid seed production of both self-pollinated (e.g., tomato; Atanassova and Georgiev 2002) and cross-pollinated (e.g., onion, Kamenetsky and Rabinowitch 2002) plants, with the consequent increase in heterozygosity, hybrid vigor and production.

Male sterility is the generic name for a variety of phenomena caused by a variety of conditions; the major ones being adverse growth conditions (environment, nutrition), diseases and mutations. The phenotypic expression of male sterility varies from the complete absence of male organs, the failure to develop normal sporogenous tissues (no meiosis), the abortion of pollen throughout its development, to the absence of anthers' dehiscence or the inability of mature pollen to germinate on compatible stigma. Genetically male sterile plants of hermaphrodite species generally maintain normal female functions (Budar and Pelletier 2001).

Male sterile clones were discovered in onion (Allium cepa L.) in 1925 (Saini and Davis 1969), and since then numerous physiological, genetic and molecular studies of microgametogenesis and male sterility in various Allium crops have been published (Havey 2002; Kik 2002), e.g., onion (Havey 2000; Engelke et al. 2003), chives (A. schoenoprasum L.; Engelke and Tatlioglu 2000), leek (A. ampeloprasum L.; Havey and Lopes Leite 1999), and bunching onion (A. fistulosum L.; Yamashita et al. 2010). In most Allium crops, male fertility is strongly affected by environment (Kamenetsky and Rabinowitch 2002).

All commercial cultivars of garlic (A. sativum L.) are completely sterile: most of them do not produce visible scapes, some produce inflorescence with a few or many topsets, but none produce viable flowers. Hence no information on microgametogenesis in this important crop is available.

Complete sterility of garlic was assumed to result from the competition for nutrients between the floral and vegetative buds in the developing inflorescence (Koul and Gohil 1970), degeneration of the tapetum (Novak 1972), degenerative-like diseases induced by mycoplasma and/or viruses (Konvicka 1973), or chromosomal deletions (Etoh 1985). In addition, the malformations in embryo sac development (Etoh 1985) or disorders in the female gametophyte (Winiarczyk and Kosmala 2009) in garlic were reported. Flower abortion or sterility were also reported in a number of Allium species, including. A. vineale L., A. oleraceum L., A. carinatum L., A canadense L., A. macrostemon Bunge, A. caeruleum Pall. It was suggested that in these species, much like in garlic (Pooler and Simon, 1994), competition for nutrients between the developing flowers and topsets might lead to floral aberrations (Etoh 1985; Mathew 1996; Kamenetsky and Rabinowitch 2002).

Recently, a number of garlic genotypes, mostly landraces from Central Asia, proved to be fertile (Etoh et al. 1988; Pooler and Simon 1993, 1994; Jenderek and Hannan 2000; Kamenetsky et al. 2005). Additionally, fundamental physiological and molecular studies enabled the induction of flowering and restoration of fertility by environmental manipulations (Kamenetsky et al. 2004). Seeds were produced from selected genotypes under a wide range of climatic conditions (Jenderek and Hannan 2004; Jenderek and Zewdie 2005; Kamenetsky et al. 2005; Shemesh et al. 2008), thus facilitating the broadening of the genetic variation, and enabling genetic studies, and breeding (Etoh and Simon 2002; Simon and Jenderek 2004; Kamenetsky 2007). However, recent observations showed that bolting and flowering garlic genotypes vary in fertility and in their ability to produce viable pollen, probably due to disorders in floral organogenesis. The understanding of gametogenesis, the fertilization processes and embryology are expected to facilitate hybridization, the development of viable seed and conventional breeding in garlic.

### RELATED BACKGROUND ART

WO199847331
WO2010007059
Van der Meer Q P, Van Bennekom J L (1969) Effect of temperature on the occurrence of male sterility in onion. Euphytica 18:389-394.

### SUMMARY OF THE INVENTION

According to an aspect of some embodiments of the present invention there is provided a male sterile garlic plant (Allium sativum), wherein a male-sterility of the plant is nuclear encoded.

According to an aspect of some embodiments of the present invention there is provided a male sterile garlic plant characterized by anther degeneration in closed flower buds at stage 2-3 of development.

According to an aspect of some embodiments of the present invention there is provided a garlic plant obtainable from seeds as deposited at the NCIMB Ltd. Crabstone Estate. Bucksburn, Aberdeen AB21 9YA, with deposit number YYY.

According to some embodiments of the invention, the male-sterility of the plant is cytoplasmic genetic male sterility.

According to some embodiments of the invention, the male-sterility of the plant is cytoplasmic male sterility.

According to some embodiments of the invention, the male-sterility of the plant is nuclear encoded.

According to an aspect of some embodiments of the present invention there is provided a male sterile garlic plant, wherein a male-sterility of the plant is environmentally-induced.

According to some embodiments of the invention, the plant exhibits visually normal development of androecium (male organs) and gynoecium (female organs), but most pollen grains are not viable.

According to some embodiments of the invention, the environmentally induced male sterility is thermosensitive.

According to some embodiments of the invention, the environmentally induced male sterility is photosensitive.

According to some embodiments of the invention, the environmentally induced male sterility is humidity-sensitive.

According to some embodiments of the invention, the male sterile garlic plant is female fertile.

According to some embodiments of the invention, the male sterile garlic plant is characterized by tapetum degeneration at late stages of pollen development.

According to some embodiments of the invention, the male sterile garlic plant is characterized by having no functional microspores.

According to an aspect of some embodiments of the present invention there is provided a hybrid garlic plant having the male sterile garlic as an ancestor.

According to an aspect of some embodiments of the present invention there is provided a method of producing a hybrid garlic plant, the method comprising:

(a) providing a first garlic plant as described herein;

(b) providing a second garlic plant that is male fertile; and

(c) crossing the first garlic plant with the second garlic plant, thereby producing a hybrid garlic plant.

According to an aspect of some embodiments of the present invention there is provided a method of producing a hybrid plant, the method comprising:

(a) providing a first plant;

(b) providing a second plant that is male fertile; and

(c) crossing the first garlic plant with the second plant, thereby producing a hybrid plant.

According to some embodiments of the invention, the second plant is of the Allium genus.

According to some embodiments of the invention, the second plant is selected from the group consisting of onion, leek and chives.

According to an aspect of some embodiments of the present invention there is provided a method of producing seeds, the method comprising:

(a) growing the hybrid plant;

(b) harvesting seeds from the hybrid plant.

According to some embodiments of the invention, the method further comprises selecting for a garlic plant following step (c) that has a male sterility trait.

According to some embodiments of the invention, the selecting is effected phenotypically.

According to an aspect of some embodiments of the present invention there is provided a method of growing a garlic plant, the method comprising somatically reproducing the garlic plant from a tissue, cell or protoplast culture derived from the male sterile garlic plant described herein or hybrid plant as described herein.

According to an aspect of some embodiments of the present invention there is provided a method of vegetatively propagating a garlic plant comprising:

(a) providing a clove of the garlic plant described herein;

(b) transferring the clove to a growth medium; and

(c) allowing the clove to grow into a plant.

According to an aspect of some embodiments of the present invention there is provided a method of inducing male sterility in a garlic plant, the method comprising subjecting the garlic plant to environmental conditions which induce male-sterility in the plant while maintaining female fertility, thereby inducing male sterility in the garlic plant.

According to some embodiments of the invention, the conditions which induce male-sterility in the plant are selected from the group consisting of temperature-inducing conditions, humidity-inducing conditions and light-inducing conditions.

According to an aspect of some embodiments of the present invention there is provided a garlic hybrid seed or hybrid plant obtainable by the method described herein.

According to an aspect of some embodiments of the present invention there is provided a male sterile garlic plant obtainable from growing the seed described herein.

According to an aspect of some embodiments of the present invention there is provided a plant part of the garlic plant described herein.

According to some embodiments of the invention, the plant part is selected from the group consisting of leaf, pollen, ovule, embryo, root tip, anthers, flowers, seeds, seed coat, stem, bulb, clove or cell or tissue of any thereof.

According to some embodiments of the invention, the plant part is a bulb.

According to an aspect of some embodiments of the present invention there is provided a garlic seed obtainable from a garlic plant described herein.

According to an aspect of some embodiments of the present invention there is provided a processed product comprising the plant part described herein.

According to an aspect of some embodiments of the present invention there is provided a sample of representative seeds of a male sterile garlic plant, wherein the sample has been deposited under the Budapest Treaty at the NCIMB under NCIMB YYY (91).

According to an aspect of some embodiments of the present invention there is provided a sample of representative seeds of a male sterile garlic plant, wherein the sample of the male sterile garlic plant has been deposited under the Budapest Treaty at the NCIMB under YYY (91).

According to an aspect of some embodiments of the present invention there is provided a sample of representative seeds of a male sterile garlic plant, wherein the sample has been deposited under the Budapest Treaty at the NCIMB under NCIMB YYY (44).

According to an aspect of some embodiments of the present invention there is provided a sample of representative seeds of a male sterile garlic plant, wherein the sample of the male sterile garlic plant has been deposited under the Budapest Treaty at the NCIMB under YYY (44)

According to some embodiments of the invention, the male-sterility of the plant is nuclear encoded.

According to some embodiments of the invention, the male-sterility of the plant is cytoplasmic genetic male sterility.

According to some embodiments of the invention, the male sterile garlic plant is characterized by anther degeneration in closed flower buds at stage 2-3 of development.

### BRIEF DESCRIPTION OF THE DRAWINGS

Some embodiments of the invention are herein described, by way of example only, with reference to the accompanying drawings/images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a graph showing the temperature conditions in Bet Dagan, Israel, during the observations, experiments and studies of pre-anthesis and anthesis stages of garlic genotypes (April-June 2009 and 2010).

FIGS. 2A-C are images of the inflorescence structure of fertile garlic. FIG. 2A—Complete inflorescence. Bar=3 mm; FIG. 2B—Longitudinal sections of inflorescence. The oldest flower buds at the top. Bar=3 mm; FIG. 2C—acropetal differentiation of flowers in a single cyme. Bar=1.5 mm

FIGS. 3A-J are images showing the stages of development of garlic flower from spathe opening to senescence, Bar=1 mm

FIGS. 4A-D are images showing the gradual maturation of anthers in garlic, from closed sacs (FIG. 4A) to stomium opening (FIG. 4B) and pollen release (FIGS. 4C-D). Bar=150 μm.

FIGS. 5A-C are images showing the gradual maturation of the stigma in garlic, from immature to receptive. Bar=25 μm. FIG. 5A—Beginning of anthesis. Papillae on smooth stigma surface are visible (stage 4, FIGS. 2A-C). FIG. 5B—Non-receptive stigma during stages 6 and 7 (FIGS. 2A-C), when pollen sheds; papillae at the stigma surface become wrinkled. FIG. 5C—Style elongates beyond the anthers' tops (stages 8 and 9, FIGS. 2A-C), stigma is receptive; papillae are wrinkled and misshapen.

FIGS. 6A-L are images showing microsporogenesis in fertile and male-sterile garlic genotypes. FIG. 6A—Cross-section of a garlic anther reveals the interior of the pollen sac. Two cells of sporogenic tissue (st) are visible. Bar=10 μm. FIG. 6B—First prophase of the MMC. Bar=10 μm. FIG. 6C—First anaphase of the MMC. Bar=10 μm. FIG. 6D—First telophase of the MMC. Bar=10 μm. FIG. 6E—Dyad. Bar=10 μm. FIG. 6F—Second telophase. Bar=10 μm. FIG. 6G—End of the second meiotic division: a tetrad enclosed by the callose wall (c) is visible. Bar=10 μm. FIG. 6H—Degradation of the callose wall resulting in tetrad separation. Bar=8 μm. FIG. 6I—Mitosis of microspore, each cell containing a single nucleous. Bar=6 μm. FIG. 6J—A mature pollen grain of the fertile genotype #1000 containing a vegetative (vc) and generative cell (gc). Bar=6 μm. FIG. 6K—A mature pollen grain of male-sterile genotype #3028, containing a vegetative (vc) and generative cell (gc). Bar=6 μm. FIG. 6L—Pollen grains with degenerated cytoplasm, male-sterile genotype #3028. Bar=15 μm.

FIGS. 7A-F are images showing anther and pollen development in fertile and male-sterile garlic genotypes. FIG. 7A—Cross-section of a pollen sac, containing MMC in the first prophase. Epidermis (e), endothecium (et), tapetum (t) and middle layer (ml) are visible. Bar=50 μm. FIG. 7B—Tetrad development in the pollen sacs. Four pollen sacs and the central filament are visible. Bar=50 μm. FIG. 7C—A pollen sac with pollen grains (pg). Epidermis (e), endothecium with ligno-cellulosic thickenings (et), middle layers are missing and the tapetum degenerates (t). Microspores are visible inside of the loculus. Bar=50 μm. FIG. 7D—A wide open stomium (s) allows the release of mature pollen. Bar=100 μm. FIG. 7E—Pollen sac containing aborted pollen grain of the male-sterile genotype #3028. Bar=50 μm. FIG. 7F—Pollen sac of male-sterile genotype #2000. Secretory tapetum disintegrating (arrows), clear gap between the tapetum and the middle layer and degenerating and aborting pollen grains inside loculus are visible. Bar=100 μm.

FIGS. 8A-B are images showing pollen germinability of two garlic genotypes. Bar=40 μm. FIG. 8A—Male-sterile #2000—low (≦5%) germination observed. FIG. 8B—Fertile #87—a high rate of pollen germination.

FIGS. 9A-D are images showing a variety of morphological and functional disorders in garlic flowers. Bar=1 mm FIG. 9A—Male sterility type 1, observed in genotypes #3028, 2000, 96, L, L13 and L15. Anthers turn from green to yellow, filaments do not elongate, withering occurs early in the floral bud development at stage 2-3. FIG. 9B —Male sterile type 2, observed in genotypes #3028, 44, 91, 96. Anthers degenerating in the closed flower buds at stage 2-3 and turning yellow (arrow). Styles elongate and the stigma is receptive. FIG. 9C—Male sterility type 3, observed in genotypes #2000, 3027. In morphologically normal anthers, pollen is not vital. Filaments' elongation is normal, but one anther turns yellow, and its pollen sacs remain closed (arrow). The style elongates, the stigma is receptive. FIG. 9D—Male sterility type 3, observed in genotypes #2000, 3027. In morphologically normal anthers pollen is not vital. All anthers are purple, but pollen sacs of some anthers (arrow) do not open. The style elongates normally and the stigma is receptive.

FIGS. 10A-F are images showing comparison between Coomassie Blue-stained 2-D protein maps of protein extracts from the anthers of fertile and male-sterile garlic genotypes. FIG. 10A—Fertile genotype #87, microspore stage, 45 specific proteins detected. FIG. 10B—Fertile genotype #87, pollen grain stage, 84 specific proteins detected. FIG. 10C—Male-sterile genotype #3028 (Types 1 and 2), microspore stage, 52 specific proteins detected. FIG. 10D—Male-sterile genotype #3028 (Types 1 and 2), pollen grain stage, 10 specific proteins detected. FIG. 10E—Male-sterile genotype #2000 (Type 3), microspore stage, 47 specific proteins detected. FIG. 10F—Male-sterile genotype #2000 (Type 3), pollen grain stage, 112 specific proteins detected.

FIGS. 11A-D are images showing the comparison between Coomassie Blue-stained 2-D protein maps of protein extracts from the anthers of fertile, male-sterile and completely sterile garlic genotypes at the final stages of the anthers' development. FIG. 11A—Fertile genotype #87, 118 specific proteins detected prior to the anther's opening. FIG. 11B —Male-sterile genotype #3028 (Types 1 and 2), 59 specific proteins detected at pre-anthesis stage. FIG. 11C—Completely sterile genotype #L (Type 1), 329 specific proteins detected in the anthers prior to the withering of the flower buds. FIG. 11D—Completely sterile genotype #L11 (Type 1), 331 specific proteins detected in the anthers prior to the withering of the flower buds.

FIG. 12 is a scheme of possible barriers in the development of garlic flowers depicting three types of male sterility.

### DESCRIPTION OF SPECIFIC EMBODIMENTS OF THE INVENTION

The present invention, in some embodiments thereof, relates to male sterile garlic plants, hybrid offspring of same and methods of generating and using same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Commercial cultivars of garlic (Allium sativum) do not produce viable flowers and seed; hence information on microgametogenesis and genetic knowledge of this important crop is unavailable. Recently, physiological studies enabled flowering and fertility restoration in garlic bolting genotypes by environmental manipulations, thus broadening of the genetic variation and facilitating genetic studies.

Whilst reducing the present invention to practice, the present inventors selected and identified specific garlic genotypes varying in their fertility traits. Morphological and anatomical studies revealed completely fertile genotypes, as well as variation in anther and pollen development and disruption of the male organs and gametes at different developmental stages. Three types of garlic plant sterility were observed, including complete sterility (type 1), male sterility (type 2) and environmentally-induced male-sterility (type 3).

The availability of selected male sterile and male fertile garlic genotypes enables pollination and fertilization of the male sterile plants by male fertile plants, and thus opens the way for genetic studies, utilization of genetic variation and efficient production of hybrid seed. Much like other cross-pollinated plants, garlic is expected to benefit from heterozygosity and hybrid vigor. Therefore, the development of reliable markers for male sterility is of considerable benefit.

Thus, according to an aspect of the invention there is provided a male sterile garlic plant (Allium sativum), wherein a male-sterility of the plant is nuclear encoded or cytoplasmic genetic male sterility.

According to another aspect of the invention there is provided alternatively or additionally a male sterile garlic plant characterized by anther degeneration in closed flower buds at stage 2-3 of development.

According to a specific embodiment, the male-sterility of the plant is cytoplasmic genetic male sterility.

According to a specific embodiment, the male-sterility of the plant is cytoplasmic male sterility.

According to a specific embodiment, the male-sterility of the plant is nuclear encoded.

According to another aspect of the invention there is provided alternatively or additionally a male sterile garlic plant, which is environmentally-induced.

According to a specific embodiment, the anthers of the plant are morphologically normal (e.g., as in genotype 1000 or 87) but pollen is sterile. In other words, the plant exhibits visually normal development of both androecium (male organs) and gynoecium (female organs), but most (above 50%, e.g., 60-100%, 70-100%, 70-90%) pollen grains are not viable, as determined in for example a functional assay e.g., germination assay further described hereinbelow.

It is appreciated that environmentally induced male sterility may be thermosensitive i.e., induced by high or low temperatures, photosensitive (light intensity, light spectrum and photoperiodic response) or dryness (induced by low air humidity e.g., below 50%), or a combination of same.

According to yet another aspect of the invention there is provided alternatively or additionally a male sterile garlic plant obtainable from seeds as deposited at the NCIMB Ltd. Crabstone Estate. Bucksburn, Aberdeen AB21 9YA, on YYY with deposit number YYY (genotype 91).

According to yet another aspect of the invention there is provided alternatively or additionally a male sterile garlic plant obtainable from seeds as deposited at the NCIMB Ltd. Crabstone Estate. Bucksburn, Aberdeen AB21 9YA, on YYY with deposit number YYY (genotype 44).

Further there is provided a sample of representative seeds of a male sterile garlic plant, wherein said sample has been deposited under the Budapest Treaty at the NCIMB under NCIMB YYY (genotype 91).

Yet further there is provided a sample of representative seeds of a male sterile garlic plant, wherein said sample of said male sterile garlic plant has been deposited under the Budapest Treaty at the NCIMB under YYY (genotype 91).

Further there is provided a sample of representative seeds of a male sterile garlic plant, wherein said sample has been deposited under the Budapest Treaty at the NCIMB under NCIMB YYY (genotype 44).

Yet further there is provided a sample of representative seeds of a male sterile garlic plant, wherein said sample of said male sterile garlic plant has been deposited under the Budapest Treaty at the NCIMB under YYY (genotype 44).

Deposits were mailed by FedEx on Feb. 26, 2013, tracking number 871993046237;

The plants produced from any of the above representative seeds is characterized by male-sterility that is nuclear encoded.

The plants produced from any of the above representative seeds is characterized by male-sterility that is cytoplasmic male sterility.

Alternatively, the plants produced by any of the above representative seeds is characterized by male-sterility that is cytoplasmic-genetic male sterility.

Alternatively or additionally, the plants produced from any of the above representative seeds is characterized anther degeneration in closed flower buds at stage 2-3 of development.

As used herein the term “garlic plant” or “Allium sativum” refers to any plant, line, accession, cultivar, landraces or population known under the species name. The invention is aimed to encompass all varieties of garlic.

Modern taxonomy divides the A. sativum species complex into three major groups—the common garlic group, the Longicuspis group and the Ophioscorodon group—and two additional subgroups: the Subtropical and the Pekinense (Fritsch and Friesen 2002 Evolution, domestication and taxonomy. p. 5-30. In: H. D. Rabinowitch and L. Currah (eds.), Allium crop sciences: recent advances, CAB Int., Wallingford, UK). Botanical species A. longicuspis L. and Allium sativum var. ophioscorodon Doll are considered as groups within the A. sativum species complex (Hanelt 1990; Etoh and Simon 2002; Fritsch and Friesen 2002).

Horticultural classification divides garlic complex into five horticultural groups: Rocambole, Continental, and Asiatic, which produce tall flower stalks with many small topsets, and Artichoke and Silverskin, which produce only a few large sets (bulbils) within false stems and early-maturing bulbs (Engeland, R. I. 1991. Growing Great Garlic: The Definitive Guide for Organic Gardeners and Small Farmers. Filaree Farms, Okanogan, Wash.; Engeland, R. I. 1995. 1995 Supplement to Growing Great Garlic. Filaree Prod., Okanogan, Wash.)

In horticultural practice, garlic cultivars are broadly classified into two main categories: hardneck and softneck. Hardneck cultivars produce a flower stalk—technically, a scape—and are often termed “topsetting” or “bolting” cultivars. Flowers, when produced, usually abort, and small bulbs—“topsets” are formed in the inflorescence. Typically, hardneck garlic cultivars produce bulbs comprising one or two whorls with four to 12-15 cloves surrounding the flower stalk. Softneck cultivars do not produce a flower stalk, and the bulb generally contains a number of whorls with 10 to 50 cloves.

As used herein, the term “accession” refers to a genetically heterogeneous collection of plants sharing a common genetic derivation.

As used herein, the term “variety” or “cultivar” means a group of similar plants that by structural, morphological, physiological or genetic features and/or performance can be distinguished from other varieties within the same species/crop.

A “cultivated plant” is defined herein as a plant exhibiting agronomically desirable characteristics. The term is used herein contrast to the term “wild”, which indicates plants that are of no immediate commercial interest. i.e., plants found in natural populations or habitats, and not utilized in commercial production.

As mentioned, the garlic of the invention or the ancestral origin of the hybrids described herein is male sterile.

Male sterility indicates that a plant has no fertile pollen and hence male sterile plants are incapable of self pollination genotypes.

The term “male sterile” is used herein in its art-recognized meaning. Male sterile means the inability to form viable pollen. This may be due to pollen abortion or when the pollen is viable but it cannot reach the ovary. Thus, according to one embodiment, the pollen is viable but fertilization is impossible due to morphological or biochemical barriers.

According to a specific embodiment the male sterility is nuclear encoded or genetic male sterility.

As used herein, the term “nuclear” or “genetic” means originating from the nucleus. Nuclear sterility means that the sterile trait (or gene encoding thereto) originates from the nucleus.

Alternatively the male sterility of the garlic plants of the invention is cytoplasmic male sterility.

As used herein, the term “cytoplasmic” means originating from the cytoplasm. Cytoplasmic sterility means that the sterile trait (or gene encoding thereto) originates from the cytoplasm (mitochondrial male sterility).

Yet alternatively the male sterility is “cytoplasmic genetic”.

As used herein “cytoplasmic-genetic male sterility” refers to the sterility that is manifested by the influence of both nuclear (with Mendelian inheritance) and cytoplasmic (maternally inherited) genes.

The male sterility may be complete male sterility or partial male sterility.

According to a specific embodiment the male sterility is dominant male sterility.

As used herein, the term “dominant” refers to the relationship between alleles of a gene, in which one allele dominates the performance (phenotype) of the trait(s) coded by the same locus. In the simplest case, where a gene exists in two allelic forms (designated A and a), three combinations of alleles (genotypes) are possible: Aa, AA, and aa. If AA and aa individuals (homozygotes) show different forms of the trait (phenotype), and Aa individuals (heterozygotes) show the same phenotype as AA individuals, then allele A is said to dominate or be dominant to or show dominance to allele a, and a is said to be recessive to A.

Dominant male sterility in the present invention indicates that all (100%) of the F1 offspring is male sterile.

According to another embodiment of the invention, about 10% to about 80% of a F1 offspring is male sterile. According to another specific embodiment, about 20% to about 70% is male sterile. According to another specific embodiment, about 40% to about 50% is male sterile. Such values indicates a multi-gene trait.

As used herein, the term “allele(s)” refers to alternative forms of a gene, all of which alleles relate to at least one trait or characteristic. Since garlic is a diploid plant (16 chromosomes), two alleles of a given gene occupy corresponding loci on a pair of homologous chromosomes. However, the invention also relates to hybrid plants which may be of alternative ploidy such as with a tetraploid plant such as leek (having 32 chromosomes). In such cases the hybrid is mostly triploid (e.g., 24 chromosomes). As used herein the term “gene” refers to a hereditary unit consisting of a sequence of DNA that occupies a specific location on a chromosome and that contains the genetic instruction for a particular characteristic or trait in an organism.

As used herein the term “locus” refers to the position that a given gene occupies on a chromosome of a given species.

As used herein, the term “heterozygous” means a genetic condition existing when different alleles of the same gene reside at corresponding loci on homologous chromosomes.

As used herein, the term “homozygous” means a genetic condition existing when identical alleles of the same gene reside at corresponding loci on homologous chromosomes.

As used herein, the term “offspring” means any product of a cross between individuals or specific lines. Offspring includes but is not limited to seed and/or plant.

In general, cytoplasmic sterility is inherited from the female plant. Establishing that the male sterility is nuclear encoded male sterility can be done as follows:
hybridization between a plant with (assumed) nuclear encoded dominant male sterile and a male fertile plant to produce offspring; in the offspring, selecting for nuclear encoded dominant male sterile plants (F1); crossing the selected nuclear encoded dominant male sterile plant with the female fertile plant to produce offspring. In addition to classic approach (as above), molecular methods can be applied.

According to an embodiment, the male sterile plant is characterized by anther degeneration in closed flower buds at stage 2-3 (e.g., stage 2, see Table 1) of development.

As used herein the phrase “anther degeneration” refers to withering and shriveling of the anthers at pre-mature stages of development, resulting in abnormality in pollen differentiation and viability.

The present inventors have defined the major steps in flower development in fertile garlic plants and these are provided infra.

TABLE 1
duration of developmental stages in fertile Allium sativum plants*

| Stage | Description of the flower bud and flower | Duration (days) |
|---|---|---|
| 1 | Closed green tepal, green anthers | >(−14) |
| 2 | Closed pink tepals, green anthers | (−14) to (−10) |
| 3 | Closed purple tepals, anthers turn pink | (−8) to (−6) |
| 4 | Anthesis: 3-4 mm tepals, anthers turn purple | 0 |
| 5 | Anthers longer than tepals | 1 |
| 6 | Pollen shedding | 2 |
| 7 | Most anthers open and pollen shed | 3 to 4 |
| 8 | Style above anthers' tops, stigma receptive | 4 to 5 |
| 9 | Anthers wither, stigma become receptive | 6-7 |
| 10 | Flower withers | 8 |

*based on genotypes 1000 and 87, as described in Table 2 below

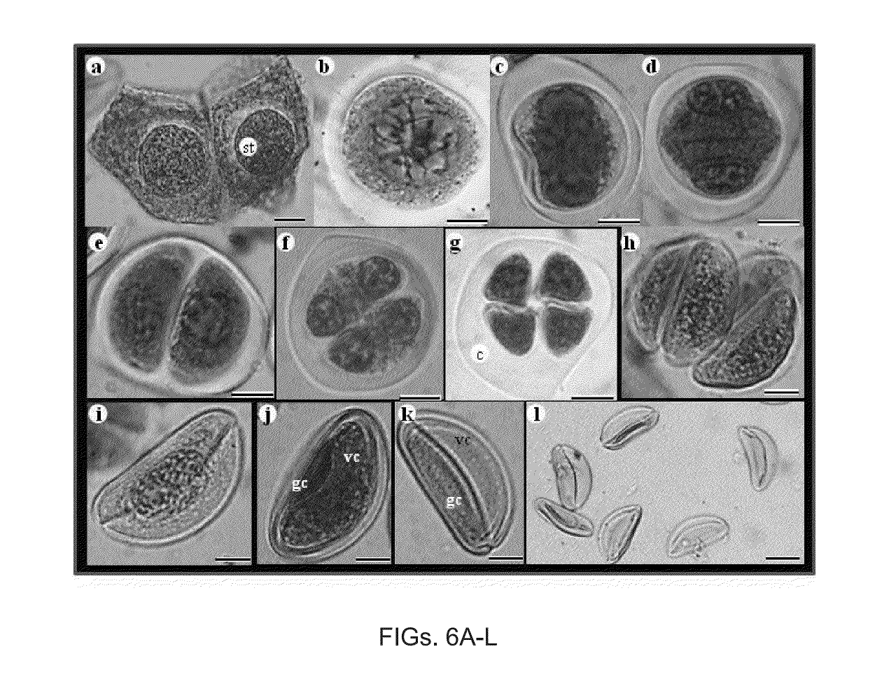

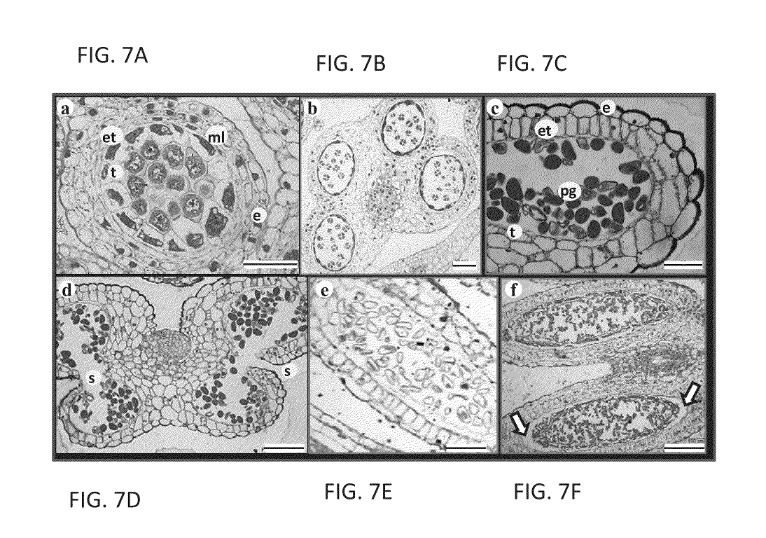

Thus, according to a specific embodiment, the pollen development discontinues after the differentiation of the vegetative and generative cells (first mitosis) (FIG. 6k), as in genotypes #3028, 44, 91 and 96. Abnormal structures or dysfunctional morphology of anthers are visible during the early stages of floral development (stages 2-3, see Table 1 above). At anthesis, the anthers turn from green to yellow and remain closed, and the degenerated microspores become empty (FIGS. 6l, 7e). Such a plant is also referred to herein as being a type 2 plant.

According to a specific embodiment, the garlic plants of the invention comprise female fertile organs.

According to a further specific embodiment, the garlic plant is characterized by tapetum degeneration at late stages of pollen development (stages 2-3, Table 1).

According to a further specific embodiment, the garlic plant characterized by having no functional microspores, essentially meaning that while the flower comprises microspores these are found non-functional in a functional assay as described below.

Types 1 and 3 as further described hereinbelow are also contemplated according to the present teachings.

As used herein “a type 1 plant” refers to a garlic plant which comprises abnormal structures or dysfunctional morphology of anthers occurring during the early stages of floral development (stages 2-3) with the consequent floral sterility. This type was evident in all differentiated flowers of the genotypes #L, L13 and L15 grown in Poland, as well as in some flowers of #3028, 2000, 44, 91 and 96 grown in Israel, see Examples section which follows. Microgametogenesis is already retarded at the one- or two-nuclei microspore stages of development, and gametogenesis is never completed. The female organs of these flowers are not functional, therefore the flowers are completely sterile and eventually flower buds wither at the pre-anthesis stage.

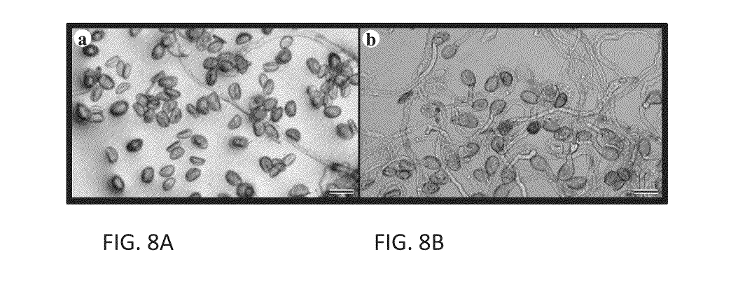

As used herein “a type 3 plant” refers to garlic plants in which the anthers morphology seems normal, and pollen shedding occurs. Yet, microscopic observations show degenerated pollen grains inside the anther. Following shedding, germination rates are rather low, less than about 20%, 10% or 5% as in genotypes #2000 and 3027 (FIGS. 7f, 8a). Female organs of the same flowers are fertile.

Garlic plants with type 3 male sterility exhibit visually normal development of both androecium (male organs) and gynoecium (female organs), but most pollen grains are not viable. Similar occurrence termed ‘incomplete male-sterility’ was described in bulb onion (Van der Meer Q P, Van Bennekom J L (1969) Effect of temperature on the occurrence of male sterility in onion. Euphytica 18:389-394).

High and low temperatures at the early stages of onion microgametogenesis resulted in poor pollen fertility. According to a specific embodiment, the male sterility is induced by high or low temperatures. The mean daily temperature during breakup of pollen tetrads markedly influences the amount of pollen produced. Similarly, high temperature during the pre-anthesis and anthesis stages of garlic flowers may adversely affect pollen fertility. It is contemplated that temperatures of 28-35° C. or higher or dry air (relative humidity of 40-50%), or combination of both, during pre-anthesis stage (10-12 days before flowering) negatively affect pollen fertility in garlic and markedly reduce pollen germination.

Qualification of the temperatures can be done under fully controlled environmental conditions (phytotron) revealing the effect of temperature regime(s) on floral development, pollen differentiation and male sterility in 3 genotypes: #87(fertile); #96(male sterile, type 2) and #2000(male sterile, type 3). The experimental layout includes initial cultivation at two temperature regimes: 16/10 and 22/16° C. (day/night, respectively). The plants are then transferred to the growth chambers with higher growth temperatures [22/16; 28/22 and 34/28° C. (day/night, respectively)] at early and late pre-anthesis stages. Phenotypic and genotypic differences are recorded, as affected by growth temperatures. Photoperiod and air humidity will be equal in all temperature regimes.

The male sterile plants of the present invention can be selected and identified as described in the Examples section which follows.

Yet various means may improve and contribute for efficient selection. Thus, according to a specific embodiment, the selection is marker assisted.

As used herein, the term “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, Random Amplification of Polymorphic DNA (RAPD) profile, single nucleotide polymorphisms (SNPs), microsatellite markers (e.g. SSRs), sequence-characterized amplified region (SCAR) markers, cleaved amplified polymorphic sequence (CAPS) markers or isozyme markers or combinations of the markers described herein which defines a nucleic acid sequence present on the genome.

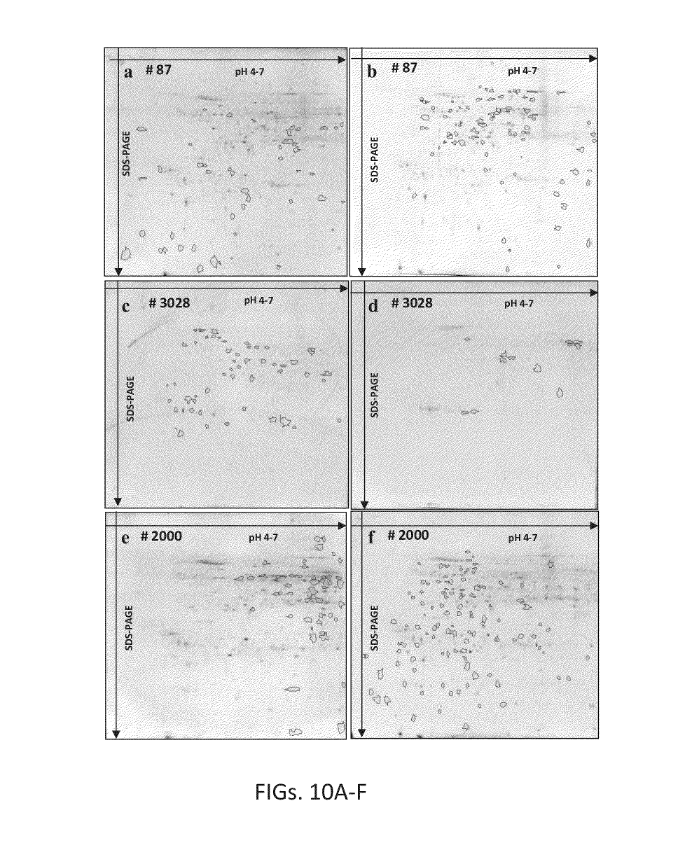

It will be appreciated that proteomic analysis can be used in selection as evident from the results presented in FIGS. 10a-f.

Alternatively, or additionally, the selection can be based on phenotypic analyses.

For example, phenological and morphological studies may be used. Buds/flowers from a number of inflorescences of each plant per genotype are tagged, and in vivo developmental morphology observations are performed daily from spathe break to flower senescence. Destructive morphological analyses of flowers are carried out at each developmental stage, under a stereoscope, and the following parameters are documented: tepals and anthers' length and color; carpel's height, width and color; style's length; time of anthesis, of filament elongation, of pollen shedding and of flower senescence.

Developmental anatomy—For anatomy and morphology studies, tissue/organs' samples are fixed in FAA solution (100% acetic acid, 40% formalin, 95% ethanol at 1:2:10 v:v:v), dehydrated in a series of ethanol concentrations of 25%, 50%, 75%, 90% and 100%, dried by liquid CO₂ (Biorad 750 critical-point dryer, England), placed on SEM discs, coated with a 10 nm gold layer and studied by scanning electron microscope (SEM JEOL, Japan) with an accelerating potential of 15 kV (Kamenetsky 1994).

For plastic embedding, tissue samples fixed in FAA are dehydrated in a graded series of ethanol as above, followed by immersion in acetone that is gradually replaced by LR-White resin (Sigma-Aldrich, St-Louis, USA). Following polymerization at 60° C. for 48-72 h, 2 μm slices are obtained using a rotary microtome (Leica RM2245). Following staining with 0.05% toluidine blue tissue slices are studied under a light microscope (Leica DMLB, Germany).

For acetocarmine staining, individual anthers are fixed and dehydrated as above, placed on a glass slide, gently squashed in 2% acetocarmine (dissolved in acetic acid 45%), and studied under a light microscope with DIC (Differential Interference Contrast, Nomarski) (Ruzin 1999).

Yet alternatively, functional analyses may be employed while selecting plants of the desired male sterility (e.g., types 2 and 3).

Thus, pollen viability and stigma receptivity assays may be employed. The number of pollen grains per anther is estimated in samples taken from a number of plants per genotype. Randomly selected newly opened anthers per sample are placed in 200 μl distilled water and vortexed for pollen release. A 10 μl diluted aliquot is studied under a light microscope, the pollen grains counted and their number per anther calculated.

Mature and dehisced anthers are squashed into a medium made up of 1% agar supplemented with 15% sucrose, and incubated in the dark for 3 h at 25° C. Pollen germination is determined under a light microscope.

Stigma receptivity is determined in flowers of each genotype by applying 10 μl DAB (Sigma Fast™ 3.3′ diaminobenzidine) solution directly onto the freshly cut stigma surface at different stages of development. The appearance of a brown color in the presence of peroxidases indicates that the stigma is receptive (Dafni et al. 2005).

Based on the above teachings the present inventors were able to identify a number of plants (clones) from each type.

A representative sample of seeds of genotypes 91 and 44 was deposited with the deposit details described above.

Seeds of garlic plants of the invention can be germinated by the following exemplary protocol. Ripened seeds are threshed, cleaned and stored under ambient conditions. The seeds may be thoroughly mixed with a fungicide e.g., Marpan (about 2%, can be obtained from Machteshim, Israel), which prevents fungal contamination of seeds and seedlings.

The seeds are stratified in moist medium (e.g., vermiculite) to prevent dehydration at 4° C. and after about 4-8 weeks are sown.

The term “plant” as used herein encompasses whole plants, ancestors and progeny of the plants and plant parts, including seeds, bulbs, cloves, shoots, stems, roots, topsets, leaves and plant cells, tissues and organs. The plant may be in any form including suspension cultures, embryos, meristematic regions, callus tissue, leaves, gametophytes, sporophytes, pollen, and microspores. Plants that are particularly useful in the methods of the invention include all plants which belong to the Allium genus, as specified above.

As used herein the phrase “tissue culture” refers to plant cells, or plant parts from garlic or hybrids thereof which can be cultured, including plant protoplasts, plant calli, plant clumps, and plant cells that are intact in plants, or part of plants, such as seeds, bulbs, bulbils, cloves, leaves, stems, pollens, roots, root tips, anthers, ovules, petals, flowers, embryos, fibers and bolls.

Plants may be regenerated from cells or tissue or organs of the plant of the invention through various procedures including but not limited to doubled haploidisation, somatic hybridization, protoplast fusion, genetic transformation, vegetative propagation using the garlic cells, pollen, protoplasts, suspension cultures, callus, basal plates, flower heads, ovules, (somatic) embryos, leaves, roots, seed and other plant parts using previously described methods such as described in Buiteveld J, Suo Y, Lookeren Campagne van M M, Creemers-Molenaar J (1998) by direct crossing or bridging. Production and characterization of somatic hybrid plants between leek (Allium ampeloprasum L.) and onion (Allium cepa L.) Theoretical and Applied Genetics 96, 765-775. Novak F J (1986) Allium. In: Evans D A Handbook of plant cell culture 4, New York, pp 419-456; WO199847331 and WO2010007059.

The availability of male sterile garlic plants paves the way to the development of new and improved cultivars propagated from seeds. Cross-pollinated plants are highly heterozygous, a preferred genotypic state with regard to seedlings' survival and plant vigor (e.g., Jones and Davis 1944). The availability of seed propagated F1 hybrids, eliminates the main ailments of clonal propagation, including carry over of pests from one generation to another, low propagation rate, voluminous storage of bulbs, rotting and sprouting, and spatial position of the transplanted cloves. The use of seeds will save the costs of vegetative propagation and spare the need for virus elimination.

Thus, according to an aspect of the invention there is provided a method of producing a hybrid garlic plant, the method comprising:

(a) providing a first garlic plant (male sterile, as described herein);
(b) providing a second garlic plant that is male fertile; and
(c) crossing said first garlic plant with said second garlic plant, thereby producing a hybrid garlic plant.

According to an alternative or additional embodiment there is provided a method of producing a hybrid plant, the method comprising:

(a) providing a first plant as described herein (e.g., the male sterile garlic, a hybrid or same e.g., interspecies or intraspecies);
(b) providing a second plant that is male fertile; and
(c) crossing said first garlic plant with said second plant, thereby producing a hybrid plant.

In another embodiment, the present invention is directed to a method of providing a male sterile plant comprising the steps of: (a) Providing a first plant that is male sterile; (b) Providing a second plant that is male fertile (c) Crossing the first and second plant to produce offspring; (d) Selecting for a plant in the offspring of step (c) that is male sterile. (e) Providing a third plant that is male fertile; (f) Crossing the selected nuclear encoded male sterile plant from step (d) with the third plant to produce offspring

As used herein, the term “hybrid plant” refers to any offspring of a cross between two genetically different individuals.

As used herein, the term “selfing” refers to a cross between genetically like individuals, often between individuals of the same offspring, or within an advanced breeding line, or established open-pollinated cultivar.

As used herein, the term “inbred” or “line” means a substantially homozygous individual

It is appreciated that the result of the above cross-pollination is the generation of a hybrid plant. As used herein, the terms “hybridization”, “hybridized” and “hybridizing” refer to both a natural and artificial process whereby the entire genome of one species, variety cultivar, breeding line or individual plant is combined intra- or interspecifically into the genome of species, variety or cultivar or line, breeding line or individual plant by crossing. The process may optionally be completed by backcrossing to the recurrent parent, as further described herein below.

According to a specific embodiment, the second plant is selected capable of producing an offspring when crossed with the first plant. Of note, crossing may be natural or man-assisted.

According to a specific embodiment, the second plant is of the species Allium sativum.

According to a specific embodiment, the second plant is not of the species Allium sativum.

According to a specific embodiment, the second plant is of the Allium genus, for instance leek (A. ampeloprasum), onion (A. cepa L.), chives (A. schoenoprasum), ramsons (A. ursinum), Chinese chives (A. tuberosum Rottier) or (A. sativum L.).

Any of the ancestral plants (parent or further ancestral origins) used for crossing can be a naïve or genetically modified plant.

Thus, any of the above methods may comprise further steps of “genetic engineering”, “transformation” and “genetic modification” which are all used herein as synonyms for the transfer of isolated and cloned genes into the DNA, usually the chromosomal DNA or genome (nuclear or non-nuclear), of another organism.

As used herein GM plants are genetically modified plants and are plants whose DNA is modified using genetic engineering techniques. In most cases the aim is to introduce a new trait to the plant which does not occur naturally in this species.

Examples include resistance to certain pests, diseases or environmental conditions, or the production of a certain nutrient or pharmaceutical agent. Genetic engineering involves the use of recombinant DNA techniques, but does not include traditional animal and plant breeding or mutagenesis, such as treating seeds or plant part with mutagens.

The development of hybrids in a plant breeding program requires, in general, the development of lines, the crossing of these lines, and the evaluation of the crosses. Most plant breeding programs combine the genetic backgrounds from two or more inbred lines or various other broad-based sources, or mutations into breeding pools from which new inbred lines are developed by selfing and selection of desired phenotypes.

Hybrids can also be used as a source of plant breeding material or as source populations from which to develop or derive new plant lines. The expression of a trait in a hybrid may exceed the midpoint of the amount expressed by the two parents, which is known as hybrid vigor or heterosis expression.

Inbred lines may for instance be derived from hybrids by using said methods as pedigree breeding and recurrent selection breeding. Newly developed inbreds are crossed with other inbred lines and the hybrids from these crosses are evaluated to determine which of those have commercial potential.

Pedigree breeding is a system of breeding in which individual plants are selected in the segregating generations from a cross on the basis of their desirability judged individually and on the basis of a pedigree record.

Recurrent selection is a breeding method based upon intercrossing selected individuals followed by continuing cycles of selection and intercrossing to increase the frequency of desired alleles in the population.

Recurrent selection may for instance be performed by backcross breeding, which involves a system of breeding whereby recurrent backcrosses are made to one of the parents of a hybrid, accompanied by selection for a specific character or characters.

The backcross is the cross of a hybrid to either of its parents. Backcrossing can for instance be used to transfer a specific desirable trait that is present in a donor plant line to another, superior plant line (e.g. an inbred line) that lacks that trait. The first step of this process involves crossing the superior plant line (recurrent parent) to a donor plant line (non-recurrent parent), that carries the appropriate gene(s) for the trait in question (e.g., the male sterile garlic). The progeny of this cross is then mated back to the superior recurrent parent followed by selection in the resultant progeny for the desired trait to be transferred from the non-recurrent parent. After five or more backcross generations with selection for the desired trait and for the germplasm inherited from the recurrent parent, the progeny will be homozygous for loci controlling the characteristic being transferred (e.g., male sterility), but will be like the superior parent for essentially all other genes. A hybrid developed from inbreds containing the transferred gene(s) is essentially the same as a hybrid developed from the same inbreds without the transferred gene(s).

A general description of breeding methods commonly used for acquiring different traits in various crops, can be found in reference books such as e.g., Allard, R. W. (1960) Principles of Plant Breeding; Simmonds, N. W. (1979) Principles of Crop Improvement; Mark J. Basset, (1986, editor), Plant Breeding Perspectives; Fehr, (1987) Principles of Cultivar Development Theory and Technique), Curah L (1986) Leek breeding: a review J Hort Sc 61: 407-415

Field crops are bred through techniques that take advantage of the plant's method of pollination. A plant is self-pollinated if pollen from one flower pollinates the same or another flower of the same plant. A plant is cross-pollinated if the pollen comes from a flower on a different plant. Plants that have been self-pollinated and selected for type for many generations become homozygous at almost all gene loci coding for the desired traits and produce a uniform population of true breeding progeny. A cross between two different such lines produces a uniform population of hybrid plants that may be heterozygous for many gene loci. A cross of two plants each heterozygous at a number of gene loci will produce a segregating population of hybrid plants that differ genetically and phenotypically and will not be uniform.

There are many important factors to be considered in the art of plant breeding, such as the ability to recognize important morphological and physiological characteristics, the ability to design evaluation techniques for genotypic and phenotypic traits of interest, and the ability to search out and exploit the genes for the desired traits in new or improved combinations.

Half-sib family: offspring of one mother plant that has been fertilized by more than one father plant either intentionally, or by open pollination.

For example, the first crossing results in the development of hybrids (garlic hybrids or inter-species hybrids) comprising male sterility component, forming a population F1 plants. Plants of the F1 population can be tested for the presence of male sterility according to the methods described above (genetic or phenotypic).

Thus, the present teachings further comprise selecting for a garlic plant following step of crossing that has a male sterility trait.

Generally, cells from the obtained F1 plants according to step (i) will have a nuclear genome which can be regarded as an intermediary genome between the first plant and the second plant (e.g., male sterile garlic and male fertile garlic or another plant of the Allium genus).

Backcrossing of male sterile BC₁ plants, i.e., the first backcross plants, with a male fertile plant can continue over any number of generations, preferable successive generations, in order to increase the amount of the genomic material of the recurrent plant in the nuclear genome of the line BC plants.

Preferably this backcrossing is continued over a number of generations (for example BC₂ to BCₙ) of the BC line. Generally, in each backcrossing, the amount of the first and second plants' genomic material will halve. In this way the use backcrossings provides a plant wherein the nuclear genome comprises substantially nuclear genetic material of the recurrent plant

End product plants with a nuclear genome of the second plant which is at least 95%, preferably 98%, more preferably 99%, and most preferably substantially 100%, further comprising the present male sterility, are suitable for obtaining other plants with male sterility properties.

According to a specific embodiment the male sterile plant or hybrid (e.g., inbred) or hybrid of same is propagated vegetatively.

Thus, there is provided a method of growing a plant (e.g., garlic or interspecies hybrid), the method comprising somatically reproducing the plant from a tissue, cell or protoplast derived from the male sterile plant described herein.

Specifically there is provided a method of vegetatively propagating a garlic plant comprising:

(a) providing a bulb of the garlic plant described herein;
(b) transferring the bulb to a growth medium; and
(c) allowing the bulb to grow into a plant.

Same can be applied on regenerating plants from tissue culture where the meristem of the plant of the invention (male sterile garlic or hybrid of same) is transferred to a growth medium and allowed to propagate.

Also provided is a garlic hybrid seed or hybrid plant obtainable by the method described herein.

Also provided is a male sterile garlic plant obtainable from growing the harvested seed.

Also provided is a plant part of the garlic plant described herein.

According to a specific embodiment the plant part is a bulb or a seed.

Thus, the present invention is directed to a male sterile seed or plant obtainable from a method according to the present invention, and to male sterile garlic plant obtainable from growing the seed according to the present invention. Furthermore, the present invention is directed to plant part derived from a male sterile garlic plants or seed or bulb according to the present invention, or obtainable from a method according to the present invention wherein the plant part is selected from the group consisting of leaf, pollen, ovule, embryo, root tip, anthers, flowers, seed, seed coat, stem, bulb, basal bulb, daughter bulbs, topsets or tissue of any thereof. More over, the present invention is directed to a regenerable cell or protoplast derived from a male sterile garlic plant or seed or bulb according to the present invention or obtainable from a method according the present invention, wherein the cell or protoplast regenerates to a garlic plant being male sterile, preferably the cell or protoplast is from a tissue selected from the group consisting of leaf, pollen, ovule, embryo, root tip, anthers, flowers, seeds, seed coat, stem, bulb. The present invention is furthermore directed to a encoded male sterile garlic plant regenerated from the cell or protoplast or plant part according to the present invention. Preferably the male sterile plant is obtainable from the method according to the present invention. In a preferred embodiment the present invention is directed to a encoded male sterile garlic plant obtainable from a bulb according to the present invention. A male sterile garlic plant according to the present invention is suitably obtained from a bulb.

The present teaching also contemplate processed products which comprise at least a plant part (e.g., cell or cell-free DNA/RNA/proteins or metabolites comprised therein) of any of the plants described herein.

The processed product can be used in the food, condiments, food supplements, pharmaceutical (e.g., garlic supplements), neutraceutical, perfume or cosmetic industry.

Examples of food products include but are not limited to garlic flakes, chopped garlic, minced garlic, granulated garlic, garlic powder and garlic oil.

Garlic supplements can be classified into four groups: garlic essential oil, garlic oil macerate, garlic chopped to any size up from cubes and flakes to powder, and garlic extract.

Garlic essential oil is obtained by passing steam through garlic. Garlic oil macerate products are made from encapsulated mixtures of whole garlic cloves ground into vegetable oil.

Garlic powder is produced by slicing or crushing garlic cloves, then drying and grinding them into powder. Garlic powder is used as a flavoring agent for condiments and food and is thought to retain the same ingredients as raw garlic and as neutraceutical and food supplement.

Garlic extract is made from whole or sliced garlic cloves that are soaked in an alcohol solution (an extracting solution) for varying amounts of time.

It is expected that during the life of a patent maturing from this application many relevant male-sterile cultivars will be developed and the scope of the term “male sterile garlic” is intended to include all such new technologies a priori.

As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

Plant Material and Growth Conditions

Garlic genotypes were obtained from the Allium Genebank in Israel and the Lublin Botanical Garden (Table 2). Four genotypes were collected in Central Asia, maintained in Israel (IGB) and treated as described in Kamenetsky et al 2004 and selected for flowering traits. Four genotypes were obtained in Israel from the seeds resulting in the previous cycles of sexual propagation. Three genotypes introduced from eastern Poland and the Marburg Botanical Garden (Germany) were grown in the Botanical Garden of Lublin, Poland.

TABLE 2
Origin and source of bolting garlic genotypes employed.

| | Source | Genotype # | Obtained from | Traits studied |
|---|---|---|---|---|
| 1 | Central | 1000 | IGB | Phenology, morphology and anatomy. |
| | Asia | | | Phylogenetic and proteomic analyses. |
| 2 | Central | 2000 | IGB | Phenology, morphology and anatomy. |
| | Asia | | | Phylogenetic and proteomic analysis |
| 3 | Central | 3027 | IBG | Phenology, morphology and anatomy |
| | Asia | | | |
| 4 | Central | 3028 | IBG | Phenology, morphology and anatomy. |
| | Asia | | | Phylogenetic and proteomic analysis |
| 5 | Israel | 44 | Propagated from a selected | Phenology, morphology and anatomy |
| | | | seedling in Israel | |
| 6 | Israel | 87 | Propagated from a selected | Phenology, morphology and anatomy. |
| | | | seedling in Israel | Phylogenetic and proteomic analysis |
| 7 | Israel | 91 | Propagated from a selected | Phenology, morphology and anatomy |
| | | | seedling in Israel | |
| 8 | Israel | 96 | Propagated from a selected | Phenology, morphology and anatomy |
| | | | seedling in Israel | |
| 9 | Poland | L | Poland | Phylogenetic and proteomic analysis |
| 10 | Germany | L13 | Poland | Phylogenetic and proteomic analysis |
| 11 | Germany | L15 | Poland | Phylogenetic and proteomic analysis |

Vegetative propagation from single bulbs guaranteed clonal uniformity. In Israel, experiments were performed in ARO, Bet Dagan. The freshly harvested garlic bulbs were cured and stored under ambient conditions from July to September in an open shed. The healthy looking propagules were then transferred to a temperature and moisture-controlled chamber at 4° C., RH=65-70% for 8 weeks. In November, healthy cloves were disinfected and planted in 200 L boxes containing 70:10:20 v/v/v 0.8 mm volcanic tuff particles: perlite: ground coconut peels, at 90 plants per square meter. The boxes were placed in a 30% shaded greenhouse covered with a 50 mesh net. Plants were fertigated regularly using “Shefer” liquid fertilizer (N:P:K=59:35:94 gL-¹, Dshanim, Israel). In Poland, the vegetatively propagated bolting garlic genotypes were grown in an open plot in the Lublin Botanical Garden.

Phenology and Morphology

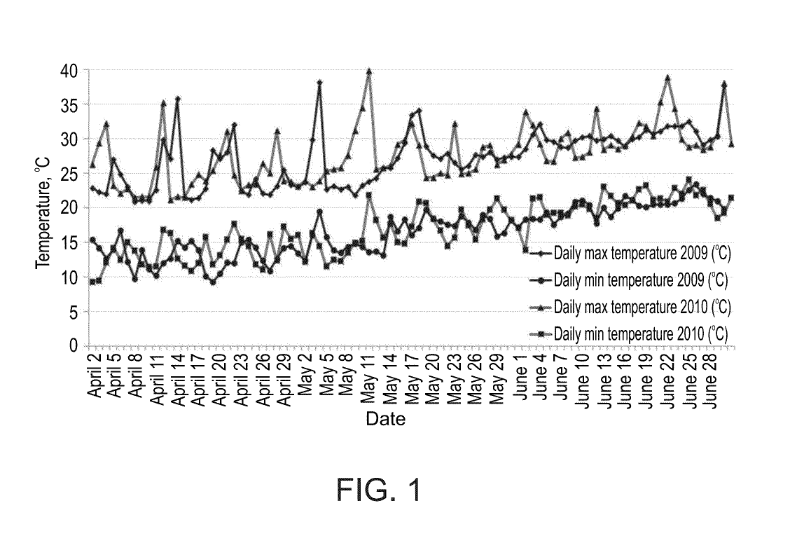

Phenological and morphological studies were performed in Israel. Maximum and minimum temperatures were recorded daily (FIG. 1). 15-20 buds/flowers from three inflorescences of each genotype were tagged, and in vivo developmental morphology observations were performed daily from spathe break to flower senescence. Destructive morphological analyses of five flowers were carried out at each developmental stage, under a stereoscope (Zeiss Stemi 2000-C, Zeiss, Germany), and the following parameters were documented: tepals and anthers' length and color; carpel's height, width and color; style's length; time of anthesis, of filament elongation, of pollen shedding and of flower senescence.

Developmental Anatomy

For anatomy and morphology studies, tissue/organs' samples were fixed in FAA solution (100% acetic acid, 40% formalin, 95% ethanol at 1:2:10 v:v:v), dehydrated in a series of ethanol concentrations of 25%, 50%, 75%, 90% and 100%, dried by liquid CO₂ (Biorad 750 critical-point dryer, England), placed on SEM discs, coated with a 10 nm gold layer and studied by scanning electron microscope (SEM JEOL, Japan) with an accelerating potential of 15 kV (Kamenetsky 1994).

For plastic embedding, tissue samples fixed in FAA were dehydrated in a graded series of ethanol as above, followed by immersion in acetone that was gradually replaced by LR-White resin (Sigma-Aldrich, St-Louis, USA). Following polymerization at 60° C. for 48-72 h, 2 μm slices were obtained using a rotary microtome (Leica RM2245). Following staining with 0.05% toluidine blue tissue slices were studied under a light microscope (Leica DMLB, Germany).

For acetocarmine staining, individual anthers were fixed and dehydrated as above, placed on a glass slide, gently squashed in 2% acetocarmine (dissolved in acetic acid 45%), and studied under a light microscope with DIC (Differential Interference Contrast, Nomarski) (Ruzin 1999).

Pollen Viability and Stigma Receptivity

The number of pollen grains per anther was estimated in samples taken from 7-10 plants per genotype. Five randomly selected newly opened anthers per sample were placed in 200 μl distilled water and vortexed for pollen release. A 10 μl diluted aliquot was studied under a light microscope (Leica DMLB, Germany), the pollen grains counted and their number per anther calculated.

Mature and dehisced anthers were squashed into a medium made up of 1% agar supplemented with 15% sucrose, and incubated in the dark for 3 h at 25° C. The specific germination protocol is provided in Shemesh et al. 2008. Pollen germination was determined under a light microscope (Hong and Etoh 1996).

Stigma receptivity was determined in 15-20 flowers of each genotype by applying 10 μl DAB (Sigma Fast™ 3.3′ diaminobenzidine) solution directly onto the freshly cut stigma surface at different stages of development. The appearance of a brown color in the presence of peroxidases indicated that the stigma is receptive (Dafni et al. 2005).

Protein Extraction and Two-Dimensional Gel Electrophoresis

Freshly harvested anthers (1 mg/plant) at different stages of development were collected from five randomly selected plants (biological replicates), placed in Eppendorf tubes and stored at −80° C. Three pooled anthers' samples were employed as technical replicates. The developmental stages of microspores and pollen grains were identified in macerated preparations of acetocarmine-stained anthers (see above). Protein extracted from the anthers according to Hurkman and Tanaka (1986). Protein concentration was determined using 2-D Quant Kit (GE Healthcare), and 2-D gel electrophoresis according to Kosmala et al. (2009) and Bocian et al. (2011). Briefly, 24 cm Immobiline DryStrip gels with linear pH range 4-7 were used for the first dimension (isoelectrofocusing, IEF), and separation on 13% polyacrylamide gels (SDS-PAGE, 1.5×255×196 mm) for the second dimension. After electrophoresis, the gels were stained with colloidal Coomassie Brilliant Blue (CBB) G-250 (Neuhoff et al. 1988). Totally separated spots were scanned using ImageScanner III (GE Healthcare) and LabScan 6.0 program (GE Healthcare) processing. Spot detection and image analyses were performed with the Image Master 2-D Platinum software (GE Healthcare). Only protein spots present in all replicates were counted. The proteomic analyses were based on the identification of qualitative differences (presence/absence of particular spots in the protein maps) between the analyzed garlic genotypes and the developmental stages.

### Example 2

### Inflorescence Structure

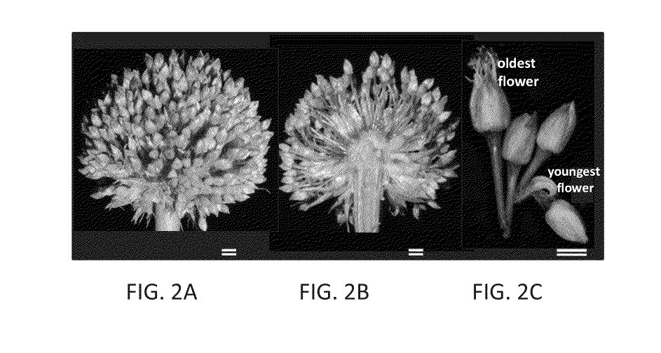

In garlic, the fully developed flowering inflorescence reaches a diameter of 3-4 cm. It consists of about 100 acropetal flower clusters (cymes), each of which is made of 5-6 flower buds and/or open flowers (FIGS. 2a, b). Cymes' development begins at the center of the umbel and the last to flower are the buds at the periphery, with respective order of seed ripening. In each cyme, flower bud formation commences at the bottom with the youngest developing at the top of the convex cluster (FIG. 2c).

Sequential Stages of Garlic Anthesis

The development of an individual flower, from spathe break to senescence, consists of 10 stages. The time line may vary between genotypes and with season.

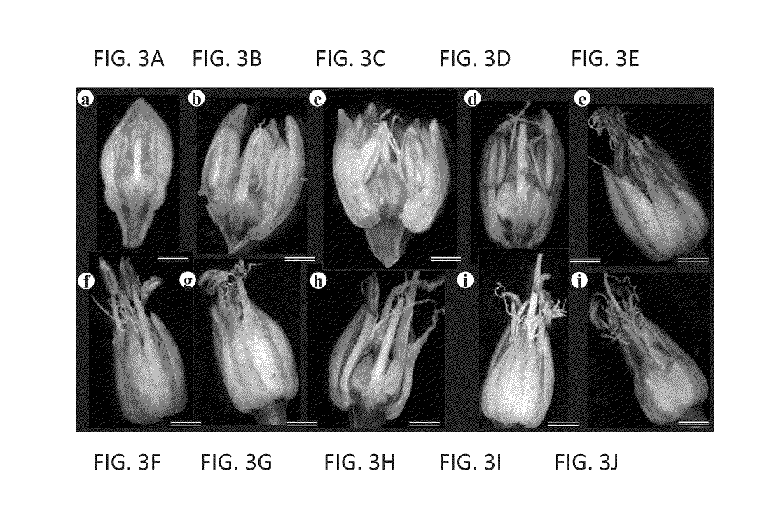

Stage 1: Flower is closed; the green anthers are completely enveloped by the green tepals. Flower bud length=2.5-3 mm (FIG. 3a).

Stage 2: Tepals elongate and their color changes from green to pink, while the anthers remain green. This stage is evident at 10-14 days prior to anthesis. Flower buds' length=3 mm (FIG. 3b).

Stage 3: The tepals' and anthers' colors turn purple and pink, respectively, on the 6-8 days prior to anthesis. Flower buds length=3-4 mm (FIG. 3c).

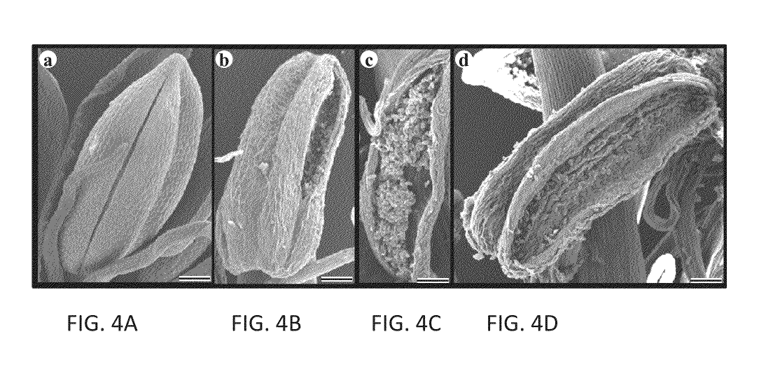

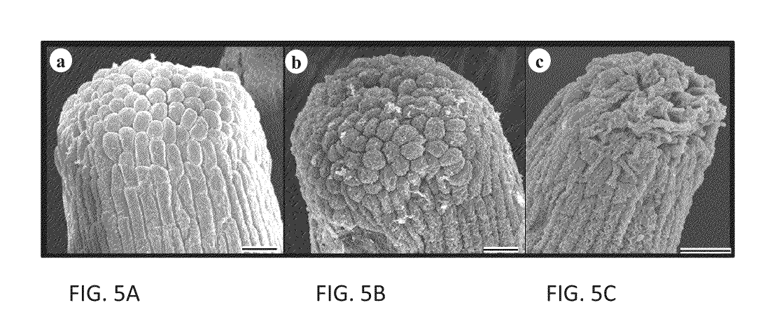

Stage 4: Anthesis occurs, tepals unfold partially, the color of the closed anthers changes to purple (FIG. 4) and stigma's surface is smooth and regular (FIG. 5a). Tepal length=3-4 mm (FIG. 3d). Commonly, under Israeli conditions, anthesis occurs in May. However, anthesis of the early- (#2000) and late (#3027) flowering genotypes were recorded at the end of April and in mid-June, respectively.

Stage 5: One day after anthesis: tepals unfold; stamens' filaments extend above the tepals and become visible (FIG. 3e). Tepal length=3-4 mm

Stage 6: Two-three days after anthesis, the gradual opening of the stomium allows for pollen shedding (FIGS. 3f, 4b). The ovary color changes from green to a dark green and purple. The filaments' and anthers' lengths are 4-5 and 0.8-1.5 mm, respectively, and their spatial position changes from vertical to horizontal (FIGS. 4b-d).

Stage 7: Pollen shedding lasts three-four days; the stigma is not receptive yet (FIG. 3g).

Stage 8: Four-five days after anthesis, pollen sacs are empty. The style elongates above the anthers' level, and reaches its final length of 6 mm (FIG. 3h). Ovary height and width are 2.2 and 1.5 mm, respectively, and ovules length is 1.25 mm. The stigma becomes receptive, while the surface's papillae turn wrinkly, and slits become visible (FIGS. 5b-c).

Stage 9: Six-seven days after anthesis, the stigma's receptivity increases concurrently with the anthers' withering (FIG. 3i).

Stage 10: The flower senescences, the tepals wither (FIG. 3j).

The time from stage 2 to 10 is ca. 20 days, while that from anthesis to senescence (stage 4-10) takes 7-8 days.

### Example 3

### Development of Viable Pollen

Pollen development from early microsporogenesis to maturation and shedding was studied in floral buds and flowers of the fertile genotype #1000. Initially the microspore mother cells (MMC) undergo differentiation (FIG. 6a) and, by the end of the first and second meiotic divisions, form dyads (FIGS. 6b-e, 7a-b) and tetrads (FIGS. 6f-g, 7b), respectively. When the tetrads' callose wall (FIG. 6g) breaks down, free microspores are released into the locular space (FIG. 6h), undergo mitotic division and reach maturation. Each mature grain contains one vegetative and one generative cell (FIGS. 6i-j). A second mitosis occurs later, at the pollen tube stage, and results in two gametes.

Anatomic studies revealed that anthers consist of typical layers of epidermis, endothecium, a middle layer, and a secretory tapetum (FIG. 7a). The latter provides nutrients to the developing pollen grain, and degenerates thereafter (FIGS. 7a-c). The tapetal and sporogenic cells contain a dense cytoplasm, and their volume increases rapidly throughout microsporogenesis. Tapetal cells contain large vacuoles and their inner wall swells slightly into the locular cavity. The tight bonding between the tapetal cells turns loose near the completion of the second meiotic division of the MMC, with the consequent separation and release of microspores, and the gradual degeneration of tapetum. The endothecium cells stretch just before dehiscence, the stomium opens and the mature pollen grains shed (FIGS. 4b-c, 7d).

### Example 4

### Developmental Aberrations of Male Organs and Male Sterility

Interruptions in male gametogenesis occurred in nine out of the 11 genotypes tested. Only genotypes #87 and 1000 produced fertile male gametes (Table 3).

TABLE 3
Flowering and fertility traits of 11 garlic genotypes

| | | | Pollen | Stigma | | | Male |
|---|---|---|---|---|---|---|---|
| | | Pollen | viability, | receptivity, | Seed | Mode of | sterility |
| Genotype | Anthesis | shedding | %* | %** | setting | sterility | type |
| 1000 | yes | yes | ·80 | 87 | yes | Fertile | — |
| 2000 | yes | yes | ″5 | 100 | yes | Male sterile | Type 3 |
| 3027 | yes | yes | ″5 | 100 | yes | Male sterile | Type 3 |
| 3028 | yes | no | 0 | 100 | yes | Male sterile | Types 1 |
| | | | | | | | and 2 |
| 44 | yes | no | 0 | 50 | yes | Male sterile | Type 2 |
| 87 | yes | yes | ·80 | 80 | yes | Fertile | — |
| 91 | yes | no | 0 | 83 | yes | Male sterile | Type 2 |
| 96 | yes | no | 0 | 50 | yes | Male sterile | Types 1 |
| | | | | | | | and 2 |
| L | no | no | 0 | 0 | no | Completely | Type 1 |
| | | | | | | sterile | |
| L13 | no | no | 0 | 0 | no | Completely | Type 1 |
| | | | | | | sterile | |
| L15 | no | no | 0 | 0 | no | Completely | Type 1 |
| | | | | | | sterile | |

*Pollen viability - percentage of germination of the pollen tubes
**Stigma receptivity - percentage of stigmas with the appearance of a brown color in the presence of peroxidases (DAB test)

It is evident that male sterile genotypes can be categorized into three main types:

1. Abnormal structures or dysfunctional morphology of anthers occur during the early stages of floral development (stages 2-3) with the consequent floral sterility. This type was evident in all differentiated flowers of the genotypes #L, L13 and L15 grown in Poland, as well as in some flowers of #3028, 2000, 44, 91 and 96 grown in Israel. Microgametogenesis is already retarded at the one- or two-nuclei microspore stages of development, and gametogenesis is never completed. The female organs of these flowers are not functional, therefore the flowers are completely sterile and eventually flower buds wither at the pre-anthesis stage.

2. Pollen development discontinues after the differentiation of the vegetative and generative cells (first mitosis) (FIG. 6k), as in genotypes #3028, 44, 91 and 96. Abnormal structures or dysfunctional morphology of anthers are visible during the early stages of floral development (stages 2-3). At anthesis, the anthers turn from green to yellow and remain closed, and the degenerated microspores become empty (FIGS. 6l, 7e). The female organs are fertile.

3. Anthers morphology seemed normal, and pollen shedding occurred. Yet, microscopic observations show degenerated pollen grains inside the anther. Following shedding, germination rates were rather low, less than 5% as in genotypes #2000 and 3027 (FIGS. 7f, 8a). Female organs of the same flowers were fertile.

Cross sections of the anthers from types 2 and 3 male-sterile plants show a clear gap between the tapetum and the middle layer at the latest stages of pollen development (FIG. 7f).

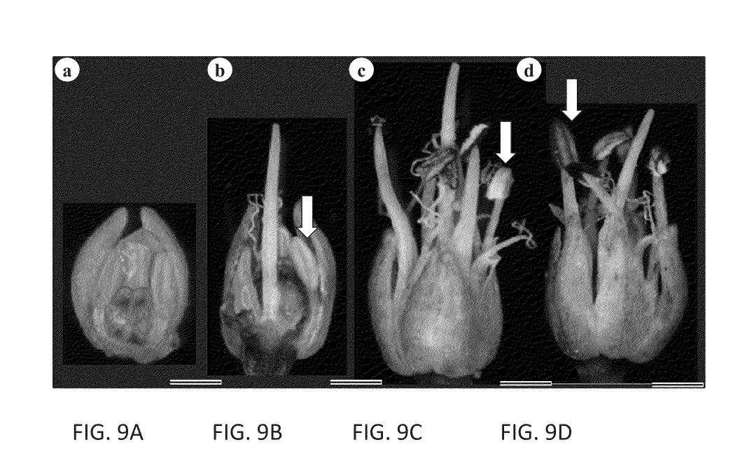

A mixture of male sterility types was obvious even in a single genotype. Hence, both type 1 and 2 were observed in genotypes #96 and #3028 (FIGS. 9a-b). A partial male sterility was also recorded, e.g., one out of the six anthers in the flowers of genotype #2000 shriveled or did not shed its pollen (FIGS. 9c-d).

### Example 5

### Pollen Viability

High pollen germinability (≧80%) was determined in two genotypes (#1000 and #87) out of the eight tested (FIG. 8, Table 3). In type 3 male sterile plants (e.g., #2000) anthers produce 100-400 non-viable pollen grains per anther, while anthers of fertile plants (e.g., #1000) contained 400-700 viable pollen grains. All pollen grains in #3028, L, L13 and L15 were aborted.

### Example 6

### Stigma Receptivity

Visual examinations of both the ovary and ovules of the flowering genotypes selected in Israel (#1000, 2000, 3027, 3028) or obtained from segregating population of seedlings (#44, 87, 91, 96), revealed no morphological deformations of the female reproductive organs, and the stigma was receptive (Table 3, above). Under the Israeli experimental conditions, stigmas become receptive only 4-5 days after anthesis and remain receptive for 3-4 days. In genotype #2000, 65% of the studied flowers had a receptive stigma immediately after style elongation (stage 8, FIG. 3), and 100% after anthers' senescence (stage 9, FIG. 3). A high percentage of stigma receptivity was recorded in genotypes #3027 and #3028 (100%), #1000 (87%) and #91 (83%).

Styles did not elongate and stigma was not receptive in any of the #L, L13 and L15 genotypes from Poland (Table 3 above).

### Example 7

### Proteomic Analysis

A comparative analysis of the protein profiles from anthers was performed. Within a pH range of 4-7, protein separations on all 2-D gels were highly repeatable and exhibited well-resolved protein maps. FIGS. 10a-f present a comparative analysis of the protein profiles for anthers from three garlic genotypes at the stages of microspores and pollen grains (FIGS. 6-7). In fertile genotypes #87, 45 and 84 specific proteins were detected at the microspore stage (FIG. 10a) and in the mature pollen grains (FIG. 10b), respectively. Anthers of the male sterile genotype #3028 (types 1 and 2) had 52 specific proteins at the microspore stage, while only 12 specific proteins were found at the stage of degenerated pollen grains (FIGS. 10c-d). Anthers of male-sterile type 3 genotype #2000, had 47 and 112 specific proteins at microsporogenesis and pollen grains, respectively (FIGS. 10e-f).

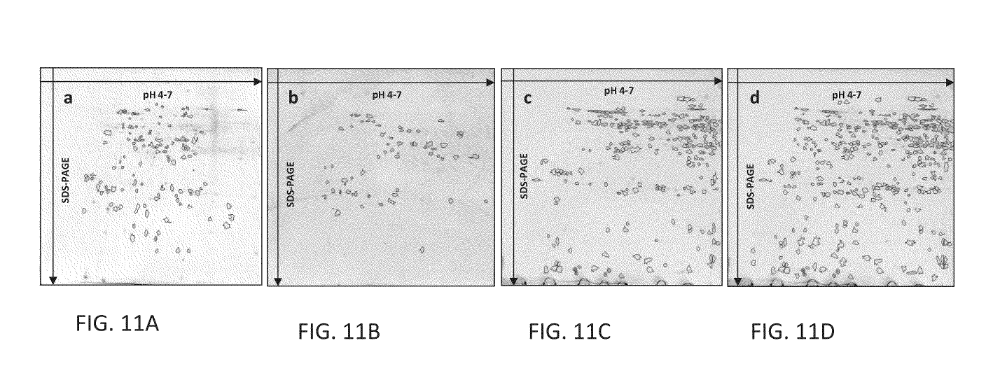

Protein maps of the anthers of fertile, male-sterile and completely sterile genotypes at the final stages of their development (FIG. 11) show the largest number of specific proteins in the completely sterile genotypes #L and L13 prior to the withering of their flower buds, in comparison with the anthers of male sterile #3028 and fertile #87 at the pre-anthesis stage.

### Example 8

### Seeds Setting

In Israel, garlic genotypes #3028, 3027, 1000, 2000, 91, 96, 44, 87 were grown in a confined space that housed active beehives. Both fertile and male sterile plants produced viable seeds (Table 3 above), thus becoming obvious that a) male-sterile plants of types 2 and 3 develop intact and functional gynoecium; b) cross-pollination is common in garlic. Genotypes #L, L13 and L15 produced neither viable flowers nor seeds during the five years of observations.

### DISCUSSION

No single comprehensive theory exists that describes and explains why some plants, including Alliums, suffer from low pollen fertility. In Allium species, it is evident that low fertility/male sterility is governed by both, genetics and environment. In bulb onion, expression of CMS reflects a nuclear-cytoplasmic incompatibility. In male-fertile onion, the tapetum nourishes microspores and degenerates after the mitotic divisions. Similarly, in early stages of microgametogenesis, CMS-S onions exhibit normal development of flowers and microspores. Abnormal development of the tapetum, however, including its breakdown at the tetrad stage; post-dyad hypertrophy followed by early autolysis, or delayed tapetal degeneration, lead to male sterility (Holford et al. 1991). In male-sterile shallot (A. cepa, Aggregatum group), microspores' interruptions occur at pre-meiosis, during the meiotic division, and/or at the first mitosis (Darlington and Haque 1955). It is thus evident that low fertility in Allium species is not phase specific, but occurs throughout the development of the androecium.

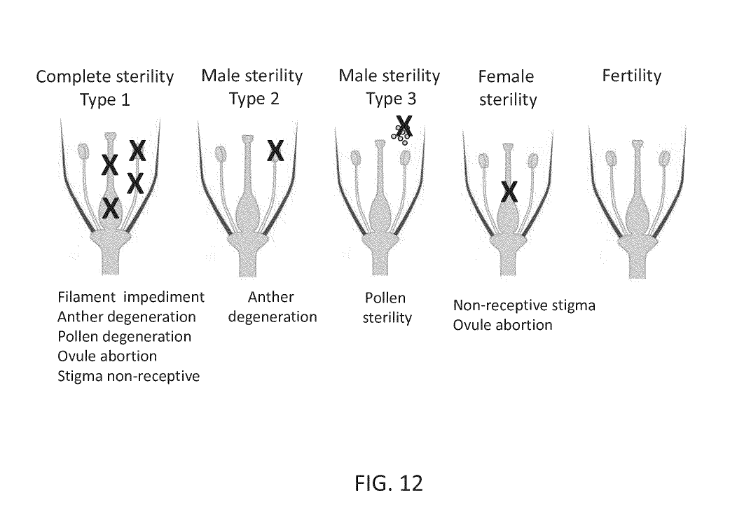

In garlic, microsporogenesis can be interrupted at various stages of development (FIG. 12). Microsporogenesis of the completely sterile plants (type 1) is already retarded at the one- or two-nuclei microspore stages. Hence no functional male gametophytes are formed.

High levels of male and female sterility were reported for the interspecific Allium hybrids between onion A. cepa and A. fistulosum L., A. roylei Steam, A. oschaninii O. Fedtsch or A. sphaerocephalon L. (McCollum 1974; Van der Meer and De Vries 1990; Keller et al. 1996). There is no evidence that A. sativum resulted from interspecific hybridization, and thus it may be concluded that the complete sterility might be caused by massive genetic interruptions through thousands of years of vegetative propagation. This process resulted in duplicated or deficient chromosomes with the consequent formation of sterile gametes, as common in some other asexually propagated bulbous crops (Etoh and Simon 2002), or strong competition between floral and vegetative buds in developing inflorescence and complete flower abortion (Etoh 1985: Kamenetsky and Rabinowitch 2002).

Garlic plants with type 2 male sterility produced no functional microspores, but are female fertile. Here, tapetal degeneration occurs after the post-mitotic formation of the generative and vegetative cells. Alternatively, development is complete and pollen reach maturity, yet viable grains remain captured in the pollen sac due to the degeneration of the anthers (FIG. 9b). Etoh and Simon (2002) argue that selection for early maturing big garlic bulbs resulted in modification of endogenous hormonal balance and the translocation of nutrients to bulbs and topsets rather than to the developing inflorescence, with the consequent sterility. In bulb onion, Heslop Harrison (1957, 1972) suggested that the competition between the developing bulb and inflorescence leads to shortages in nutrients, and that microgametogenesis is more susceptible to such a shortage than megagametogenesis. In male-sterile onion, Virnich (1967) proposed that poor microspores' nutrition due to tapetal malfunction leads to pollen degeneration. Similarly, in some type 2 male sterile garlic plants, the tapetum degenerates at the late stages of pollen development (FIG. 7e), with the consequent degeneration of the developing pollen. It is safe to suggest that this shortage in nutrients during microgametogenesis leads to pollen infertility.

Garlic plants with type 3 male sterility exhibit visually normal development of both androecium and gynoecium, but most pollen grains are not viable. Similar occurrence termed ‘incomplete male-sterility’ was described in bulb onion (Van der Meer and Van Bennekom 1969), but no convincing explanation has been provided.

High (Jones and Clarke 1943; Ockendon and Gates 1976) and low (Lichter and Mundler 1961; Van der Meer and Van Bennekom 1969) temperatures at the early stages of onion microgametogenesis resulted in poor pollen fertility. Lichter and Mundler (1961) reported that the breakup of pollen tetrads occurs on the 12th day before flowering. They suggested that the mean daily temperature during this period markedly influences the amount of pollen produced. Similarly, high temperature in Israel during the pre-anthesis and anthesis stages of garlic flowers (April, FIG. 1) may adversely affect pollen fertility.

It can be concluded that like in other Allium spp. the environment (high/low temperatures; humidity), hormonal imbalance and competition between the vegetative and reproductive organs, and/or some major genetic factors affect pollen development in garlic and its fertility.

To date, a number of researchers reported on restoration of sexual fertility in garlic (Etoh and Simon 2002; Simon and Jenderek 2004; Kamenetsky 2007). It is thus evident that with regard to flowering and fertility, the garlic genome of the genotypes served in these studies, is intact and functional. It is also evident, that under the variety of experimental conditions in the US, Europe, Israel and Japan, many garlic genotypes either do not bolt, bolt but do not produce flowers, or produce malformed and sterile flowers. Hence, it is quite likely that many of these garlic genotypes accumulated a variety of genetic interruptions (Etoh 1985), which prohibit normal development of flowers and gametes. However, the availability of male fertile plants enables pollination and fertilization of plants with fertile gynoecium, and thus opens the way for genetic studies, utilization of genetic variation and production of hybrid seed. Much like other cross-pollinated plants, garlic is expected to benefit from heterozygosity and hybrid vigor. Therefore, the development of reliable markers for male sterility is of considerable benefit.

Various molecular techniques have been used for marker-assisted selection of fertile garlic (RAPD, Etoh and Hong 2001; SNP, SSR and RAPD, Zewdie et al. 2005) and for the analysis of genetic diversity (SSR, Ma et al. 2009; AFLP, Garcia Lampasona et al. 2012). However, since morphological and/or anatomical mechanisms of male sterility in this species were not identified, molecular differentiation between fertile and male-sterile bolting genotypes has not been attempted.

Comparative analyses of protein maps revealed differences in the number of specific proteins produced by fertile and sterile genotypes. A significant difference in the proteins' profiles was also found between the early and later developmental stages of anthers and pollen development (FIGS. 10-11). Although the protein accumulation profiles cannot be regarded as a direct reflection of the corresponding gene activities, protein maps might provide information on the developmental process in garlic's reproductive organs. Proteomic studies conducted in fertile plants, e.g., in Arabidopsis (Becker et al. 2003; Chevalier et al. 2004), Lycopersicon esculentum (Sheoran et al. 2007) and rice (Kerim et al. 2003) show that the developing pollen grain contains proteins involved in a number of vital processes, including cell wall formation, regulation of transmembrane transport and the cell cycle. Significant differences were also revealed between 2D protein profiles of sterile and fertile ovules of A. sativum and A. tuberosum Rottler ex Spreng. (Winiarczyk and Kosmala 2009).

In the present study, comparable numbers of specific proteins were found at the early stages of microsporogenesis in all studied genotypes (FIG. 10), but significant differences between protein maps were evident in later stages of development. In the fertile genotypes, the number of specific proteins increased, possibly due to intense metabolism and production of viable pollen. In comparison, in Arabidopsis, the progression from proliferating microspores to differentiated pollen is characterized by large-scale repression of the early program genes and the activation of a unique late gene-expression program in the maturing pollen (Honys and Twell 2004).

In types 1 and 2 male sterile genotypes, pollen degeneration occurs at the early stages of microgametogenesis and consequently the protein number decreased with time. Ma et al. (2009) showed a significant deviation in six loci from the Hardy-Weinberg equilibrium, thus substantiating the role of genome interruption in garlic (Etoh 1980, 1985) and the consequent sterility. In the completely sterile genotypes from the Polish collection, the genome interruption that leads to defective function of both the gynoecium and androecium is rather stable, thus indicating that these plants produce specific proteins involved in microgametes programmed cell death and in pollen degeneration. In contrast, in type 3 genotypes, male sterility may well be in a transient stage and the phenotypic expression depends on the environment. Adverse conditions may lead to interference between the transcriptome and proteome with the consequent abortion of the pollen. Indeed, field observations in Israel clearly show variation in fertility in type 3 genotypes with season.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A male sterile garlic plant (Allium sativum), wherein a male-sterility of the plant is nuclear encoded.

2. A male sterile garlic plant characterized by anther degeneration in closed flower buds at stage 2-3 of development.

3. A garlic plant obtainable from seeds as deposited at the NCIMB Ltd. Crabstone Estate. Bucksburn, Aberdeen AB21 9YA, with deposit number NCIMB 42124 (91) or with deposit number NCIMB 42123 (44).

4. (canceled)

5. The male sterile garlic plant of claim 1, wherein said male-sterility of the plant is cytoplasmic genetic male sterility.

6. The male sterile garlic plant of claim 2, wherein said male-sterility of the plant is cytoplasmic male sterility.

7. The male sterile garlic plant of claim 2, wherein said male-sterility of the plant is nuclear encoded.

8. A male sterile garlic plant, wherein a male-sterility of the plant is environmentally-induced.

9. The male sterile garlic plant of claim 8, exhibiting visually normal development of androecium (male organs) and gynoecium (female organs), but most pollen grains are not viable.

10. The male sterile garlic of claim 8, wherein said environmentally induced male sterility is thermosensitive, photosensitive or humidity-sensitive.

11-12. (canceled)

13. The male sterile garlic plant of claim 1 being female fertile.

14. The male sterile garlic plant of claim 1, characterized by tapetum degeneration at late stages of pollen development or by having no functional microspores.

15. (canceled)

16. A hybrid garlic plant having the male sterile garlic of claim 1 as an ancestor.

17. A method of producing a hybrid garlic plant, the method comprising:
(a) providing a first garlic plant according to claim 1;
(b) providing a second garlic plant that is male fertile; and
(c) crossing said first garlic plant with said second garlic plant, thereby producing a hybrid garlic plant.

18. A method of producing a hybrid plant, the method comprising:
(a) providing a first plant of claim 1;
(b) providing a second plant that is male fertile; and
(c) crossing said first garlic plant with said second plant, thereby producing a hybrid plant.

19. The method of claim 18, wherein said second plant is of the Allium genus.

20. (canceled)

21. A method of producing seeds, the method comprising:
(a) growing the hybrid plant of claim 17;
(b) harvesting seeds from said hybrid plant.

22. The method of claim 17, further comprising:
selecting for a garlic plant following step (c) that has a male sterility trait.

23. (canceled)

24. A method of growing a garlic plant, the method comprising somatically reproducing the garlic plant from a tissue, cell or protoplast culture derived from the male sterile garlic plant of claim 1.

25. A method of vegetatively propagating a garlic plant comprising:
(a) providing a clove of the garlic plant of claim 1;
(b) transferring the clove to a growth medium; and
(c) allowing the clove to grow into a plant.

26. A method of inducing male sterility in a garlic plant, the method comprising subjecting the garlic plant to environmental conditions which induce male-sterility in the plant while maintaining female fertility, thereby inducing male sterility in the garlic plant.

27. The method of claim 26, wherein said conditions which induce male-sterility in the plant are selected from the group consisting of temperature-inducing conditions, humidity-inducing conditions and light-inducing conditions.

28. A garlic hybrid seed or hybrid plant obtainable by the method according of claim 17.

29. A male sterile garlic plant obtainable from growing the seed of claim 28.

30. A plant part of the garlic plant of claim 1.

31. The plant part of claim 30, wherein the plant part is selected from the group consisting of leaf, pollen, ovule, embryo, root tip, anthers, flowers, seeds, seed coat, stem, bulb, clove or cell or tissue of any thereof.

32. The plant part of claim 31, being a bulb.

33. A garlic seed obtainable from a garlic plant of claim 1.

34. A processed product comprising the plant part of claim 31.

35. A sample of representative seeds of a male sterile garlic plant, wherein said sample has been deposited under the Budapest Treaty at the NCIMB under NCIMB 42124 (91) or under NCIMB 42123 (44).

36. A sample of representative seeds of a male sterile garlic plant, wherein said sample of said male sterile garlic plant has been deposited under the Budapest Treaty at the NCIMB under NCIMB 42124 (91) or under NCIMB 42123 (44).

37-41. (canceled)
